(19) United States
(12) Patent Application Publication
(10) Pub. No.: US 2026/0102681 A1
(43) Pub. Date: Apr. 16, 2026

(54) SYSTEM FOR SUPPORTING GOLF PUTTING AND OPERATING METHOD THEREOF

(71) Applicants: Sang Hyun LEE, Seoul (KR); MATE MOBILITY CO., LTD., Seoul (KR)

(72) Inventors: Sang Hyun LEE, Seoul (KR); Seung Joon LEE, Seoul (KR); Jae Young CHOI, Gyeonggi-do (KR); Hae Young CHOI, Gyeonggi-do (KR); Jeong Beom SEO, Seoul (KR); Il Do KIM, Seoul (KR)

(21) Appl. No.: 19/346,567

(22) Filed: Oct. 1, 2025

(30) Foreign Application Priority Data
Oct. 10, 2024 (KR) 10-2024-0137710

(51) Int. Cl. A63B 69/36; A63B 60/46; A63B 71/06

(52) U.S. Cl. CPC A63B 69/3685; A63B 60/46; A63B 2060/464; A63B 2071/063; A63B 2220/12; A63B 2220/833; A63B 2225/50

(57) ABSTRACT

A putting support system includes: a putting support device embedded in a grip of a putter of a player, detecting an address position of the player on a green, and acquiring and outputting location information of a ball, which is location information at a time when the address position is detected; a server generating putting strategy information for a location of the ball based on the location information of the ball, slope of the green, and location information of a hole cup; and a terminal receiving the location information of the ball from the putting support device, transmitting the location information of the ball to the server, receiving the putting strategy information from the server, and providing the putting support device with voice guidance information determined according to the putting strategy information.

### CROSS-REFERENCE TO RELATED APPLICATION

The present application claims priority under 35 U.S.C. § 119(a) to Korean patent application number 10-2024-0137710, filed on Oct. 10, 2024, the entire disclosure of which is incorporated herein by reference.

### BACKGROUND

### 1. Technical Field

Various embodiments of the present disclosure generally relate to a putting support system and an operating method thereof, and more particularly, to a putting support system which provides a putting guide to a player using a putting support device mounted on a putter among golf clubs.

### 2. Related Art

Golf is a globally popular sport where players aim to sink a ball into a target hole. The accuracy of each shot significantly impacts the game's outcome. Particularly, putting, as the final stage of the shot, demands precision and represents the moment where the player's skill and strategy converge.

Conventional putting support systems are mostly limited to distance measurement only. Consequently, such conventional putting support systems either fail to provide real-time assistance during the putting process or lack sufficient functionality to detect user's body movements, resulting in the provision of merely limited information. Particularly, because a location of a hole cup may change in real time, there is a growing need for a system which analyzes the position and location of the player in real time to provide optimal putting information.

### SUMMARY

Various embodiments of the present disclosure are directed to a putting support system and an operating method thereof.

A putting support system according to an embodiment of the present disclosure may include: a putting support device embedded in a grip of a putter of a player, detecting an address position of the player on a green, and acquiring and outputting location information of a ball, which is location information at a time when the address position is detected; a server generating putting strategy information for a location of the ball based on the location information of the ball, slope of the green, and location information of a hole cup; and a terminal receiving the location information of the ball from the putting support device, transmitting the location information of the ball to the server, receiving the putting strategy information from the server, and providing the putting support device with voice guidance information determined according to the putting strategy information.

In an embodiment, the putting support device may include a sensor unit sensing a pressure signal transmitted to the grip of the putter, and a controller detecting the address position of the player based on the pressure signal.

In an embodiment, the controller may determine that the player has taken the address position when the pressure signal falls within a preset range.

In an embodiment, the putting support device may further include a communication unit acquiring location information of the putting support device using real-time kinematic (RTK) positioning in response to the controller determining that the player has taken the address position.

In an embodiment, the communication unit may communicate with the terminal via a short-range communication method.

In an embodiment, the putting support device may further include a speaker providing the voice guidance information to the player.

In an embodiment, the putting support device may further include a rechargeable battery providing driving power to the putting support device, and a charging terminal charging the rechargeable battery via a wired or wireless method.

In an embodiment, the putting strategy information may include information regarding putting strength and putting direction.

In an embodiment, the terminal may be mounted on a golf cart in which the player rides.

In an embodiment, the putting support system may further include a database storing player record information including previous play information of the player and golf course information including the slope of the green and the location information of the hole cup.

### BRIEF DESCRIPTION OF THE DRAWINGS

FIG. 1 is a diagram illustrating a putting support system according to an embodiment of the present disclosure;

FIG. 2 is a diagram illustrating an embodiment of a putting support device of FIG. 1;

FIG. 3 is a diagram illustrating another embodiment of the putting support device of FIG. 2;

FIG. 4 is a diagram illustrating an operation of a putting support device;

FIG. 5 is a diagram illustrating an operation of a terminal;

FIG. 6 is a diagram illustrating an operation of a server;

FIG. 7 is a diagram illustrating an operating method of a putting support system according to an embodiment; and

FIG. 8 is a diagram illustrating an example of a putting strategy provided to a player by a terminal and a putting support device.

### DETAILED DESCRIPTION

Specific structural or functional descriptions disclosed herein are merely illustrative for the purpose of describing embodiments according to the concept of the present disclosure. Embodiments according to the concept of the present disclosure may be implemented in various forms and should not be construed as being limited to the specific embodiments set forth herein.

Hereinafter, embodiments are described with reference to the accompanying drawings in order for those skilled in the art to be able to implement the technical spirit of the present disclosure.

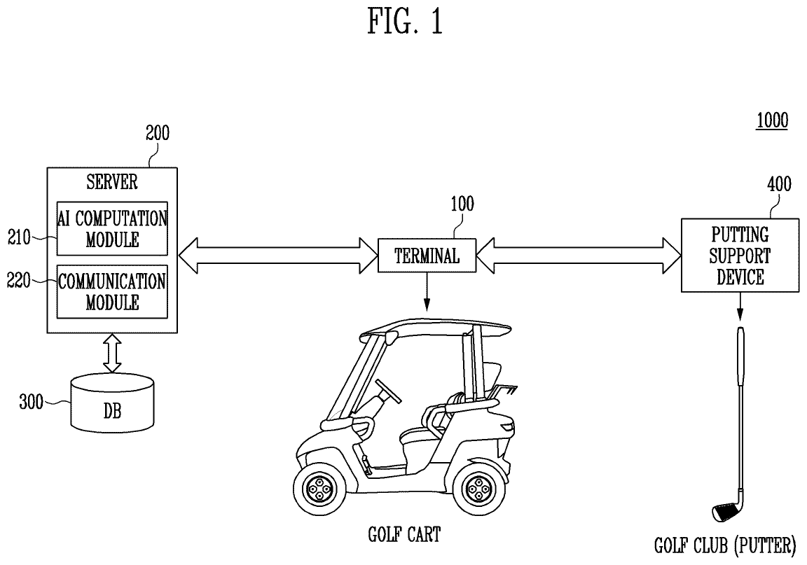

FIG. 1 is a diagram illustrating a putting support system 1000 according to an embodiment of the present disclosure.

Referring to FIG. 1, the putting support system 1000 may include a terminal 100, a server 200, a database (DB) 300, and a putting support device 400. The putting support system 1000 is network-based, where the network refers to a connection structure enabling information exchange between respective nodes such as terminals 100, servers 200, and putting support devices 400. In the present disclosure, the term “putting support system”refers to a system for supporting golf putting.

The terminal 100 may be mounted on a golf cart. The terminal 100 may be a tablet PC, but is not limited thereto, and may be implemented as various forms of electronic devices such as a smartphone, a laptop computer, or an in-vehicle infotainment system.

In an embodiment, the terminal 100 may be controlled by input from a player using the terminal 100 and may communicate with the server 200 via wireless communication. The terminal 100 is mounted on the golf cart, enabling an environment where the player may freely use the terminal 100 during golf play. The terminal 100 may perform data communication with the server 200 using a wireless communication system.

The golf cart may be a single-seater cart which each player participating in a round may drive themselves, or a multi-seater cart accommodating two or more passengers. In an embodiment, unlike the conventional 6-person golf cart which only travels on pre-set cart paths, the golf cart may enter all areas within a course, including a fairway, except for restricted areas, and move to a location of a ball of the player. Therefore, a distance a player must walk may be minimized or reduced, thereby shortening a golf playing time. In an embodiment, restricted areas may include the rough, bunker, penalty area, and green (including apron). Here, the penalty area may refer to areas where shots cannot be taken, such as lakes, streams, or drainage ditches.

When the ball of the player reaches the green, the player may use the golf cart to move to the green entrance. On the green, the player may receive putting strategy information by using the putting support device 400.

The terminal 100 provides various data related to golf play to the server 200 and may receive the putting strategy information, which is data for supporting putting, from the server 200. The terminal 100 may provide the putting strategy information to the player based on the data received from the server 200. In an embodiment, the terminal 100 may extract voice guidance information from the putting strategy information received from the server 200 or generate the voice guidance information based on the putting strategy information. The terminal 100 may provide the voice guidance information to the putting support device 400.

The terminal 100 may perform data communication with the putting support device 400. In an embodiment, because a relatively short distance of tens of meters or less may be maintained between the terminal 100 and the putting support device 400, data communication may be performed using short-range wireless communication methods such as Bluetooth, Wi-Fi, ZigBee, or Ultra-Wideband (UWB) communication method.

The putting support device 400 may be mounted on a golf club of the player. In an embodiment, the putting support device 400 may be located inside a grip of the golf club of the player. For example, the putting support device 400 may be in a form of a hardware device embedded in a shell (housing) wrapped with a grip cushion. In an embodiment, the putting support device 400 may be mounted by screwing onto a shaft of the club. In an embodiment, the shell (housing) in which the putting support device 400 is embedded may include a reinforced plastic material.

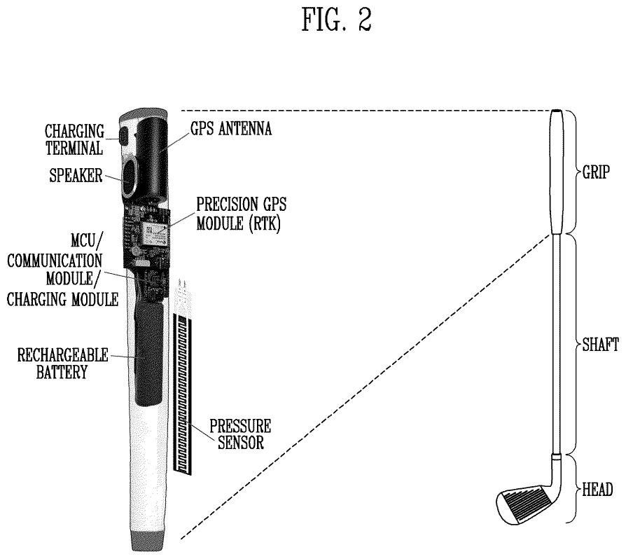

The structure and operation of the putting support device 400 are described in greater detail in the subsequent description of FIGS. 2 to 4.

The server 200 may be provided at a specific location within the golf course or at a remote location outside the golf course. The server 200 may perform data communication with the terminal 100 using a wireless communication system. The wireless communication system used by the terminal 100 and the server 200 may include a 3rd Generation Partnership Project (3GPP) network, a Long Term Evolution (LTE) network, a 5th Generation (5G) Mobile Telecommunication, a World Interoperability for Microwave Access (WiMAX) network, the Internet, a Local Area Network (LAN), a Wireless Local Area Network (Wireless LAN), a Wide Area Network (WAN), a Personal Area Network (PAN), a Bluetooth network, a satellite broadcasting network, an analog broadcasting network, a Digital Multimedia Broadcasting (DMB) network, Wi-Fi, or the like, but is not limited thereto.

The server 200 may include an AI computation module 210 and a communication module 220.

The AI computation module 210 may generate the putting strategy information using data stored in the DB 300 and data provided from the terminal 100. The AI computation module 210 may generate the putting strategy information to be provided to the player using various algorithms such as deep learning, machine learning, and a neural network circuit utilizing big data. The AI computation module 210 may provide the putting strategy information to the terminal 100 using the communication module 220.

The communication module 220 may perform data communication with the terminal 100 and the DB 300.

The DB 300 may store player record information. The player record information may include the average number of putts, putting distance information, putting trajectory information, and the like, learned from past play results of the player. In an embodiment, the information included in the player record information may further include information based on temperature, humidity, and turf type.

In an embodiment, the DB 300 may store information about the golf course. The information about the golf course may include terrain information of the golf course, par for each hole, slope of green, pin location for each hole, and turf information of the golf course. In an embodiment, the DB 300 may store data received from the server 200 or transmit data to the server 200. For example, the DB 300 may be implemented as a non-volatile memory device, such as a Solid State Drive (SSD), for storing data.

FIG. 2 is a diagram illustrating an embodiment of the putting support device 400 of FIG. 1.

Referring to FIG. 2, a golf club generally includes a head, a shaft, and a grip. The head is the part which directly strikes a ball and may have various shapes and materials depending on a type of club. The shaft connects the grip and the head and may have various materials and strengths. The grip is the part the player holds directly with their hands. The player may execute a golf swing by gripping the grip with their hands and swinging the club. The grip generally has a form to be fitted onto the shaft and may have various weights and shapes depending on the type of club. Particularly for putters, which are clubs used for putting, a relatively long and thick grip may be used compared to other clubs.

In an embodiment, the putting support device 400 may be located inside the grip.

The putting support device 400 may include a pressure sensor, a Global Positioning System (GPS) antenna, a precision GPS module (real-time kinematic (RTK) module), a Micro Controller Unit (MCU), a communication module, a charging module, a rechargeable battery, a charging terminal, and a speaker.

The pressure sensor may sense the pressure exerted by the player's hand gripping the grip through the surface of the grip. The pressure sensor converts the sensed pressure into an electrical signal and may provide the electrical signal to the MCU.

In various embodiments, although not shown in FIG. 2, the putting support device 400 may further include a button-type switch for requesting the putting strategy information. Using the button-type switch, the player may provide a control signal requesting the putting strategy information to the MCU by pressing the button-type switch at the location of the ball.

The GPS antenna may be an antenna which receives signals transmitted from GPS satellites. The MCU may calculate location information of the putting support device 400 based on the signals transmitted from the GPS satellites.

The precision GPS module (RTK module) may be a GPS module to which the concept of real-time kinematic (RTK) positioning is applied, and may acquire precise positioning results in real time at a rover by using correction values for the carrier phase of a base station (GPS base) possessing precise location information. While conventional GPS modules may incur various errors due to factors like the ionosphere, atmosphere, and satellite errors, the precision GPS module (RTK module) may compensate for the degradation in positioning accuracy caused by signal distortion and delays occurring when GPS satellite signals pass through the ionosphere, thereby reducing positioning errors to as little as 1 to 2 cm.

In an embodiment of the present disclosure, the putting support device 400 is mounted on the grip of the putter. Because the player performs the putt with the putter at the point where the ball is located, the location where the player takes an address position to perform the putt may be considered the most accurate location of the ball. Therefore, by utilizing the precision GPS module (RTK module) of the putting support device 400, the putting strategy information may be acquired in real time at the precise location of the ball.

The MCU includes a central processing unit (CPU) core, memory, and programmable input/output, and may control operations of the pressure sensor, GPS antenna, precision GPS module (RTK module), communication module, charging module, rechargeable battery, charging terminal, and speaker included in the putting support device 400.

The communication module may perform short-range communication between the putting support device 400 and the terminal 100 described with reference to FIG. 1. The communication module may provide the terminal 100 with location information acquired by the precision GPS module (RTK module), or receive voice guidance information for putting strategies from the terminal 100.

The charging module may be a module providing a function to charge the rechargeable battery. The rechargeable battery may provide the power required to drive the putting support device. In an embodiment, the charging terminal may be a wired or wireless charging terminal for charging the putting support device 400. In various embodiments, the charging module may also perform a function enabling wireless charging when a club is placed in a club holder provided on a golf cart.

The speaker may provide the player with the putting strategy information for targeting a hole cup via voice. For example, specifically, the speaker may output voice guidance information for putting strategy received from the terminal 100.

The various components included in the putting support device 400 shown in FIG. 2 are merely examples and do not limit the arrangement or function of the components included in the putting support device 400.

For example, the putting support device 400 may utilize an accelerometer or gyroscope in addition to the pressure sensor to detect the address position and movements of the player. Furthermore, more precise position analysis may be possible using various sensors. In an embodiment, an accelerometer may measure the swing speed and direction of the player and provide more accurate putting information.

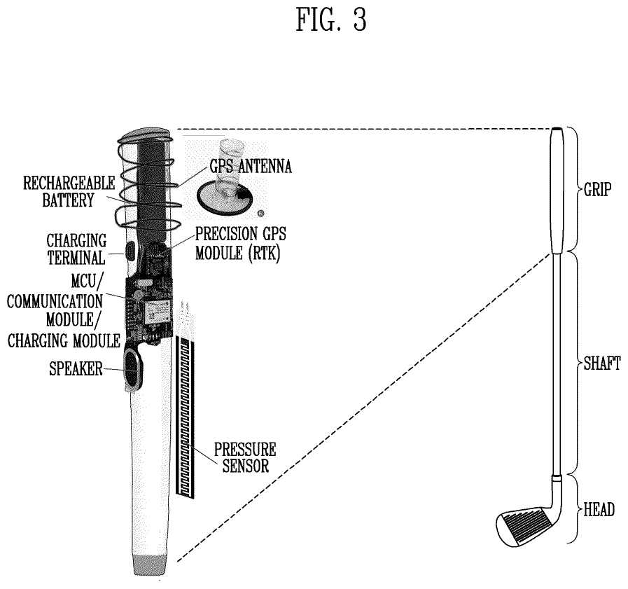

FIG. 3 is diagram illustrating another embodiment of the putting support device 400 of FIG. 2.

The difference between the embodiment shown in FIG. 3 and the embodiment shown in FIG. 2 may be the form of the GPS antenna. The GPS antenna may be mounted on a shell inside the grip as a modular component, as in FIG. 2, or the GPS antenna may be arranged coiled in a circular shape inside the grip. Furthermore, the positions of the speaker, charging terminal, and rechargeable battery may also be arranged differently from the embodiment shown in FIG. 2, as shown in FIG. 3.

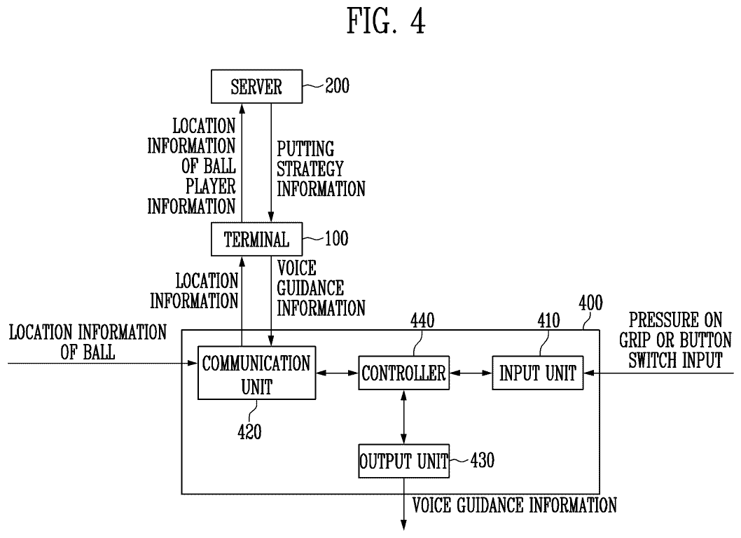

FIG. 4 is a diagram illustrating an operation of the putting support device 400.

Referring to FIG. 4, the putting support device 400 may include an input unit 410, a communication unit 420, an output unit 430, and a controller 440.

The input unit 410 may receive an input requesting the putting strategy information (voice guidance information). The input unit may be implemented as a button switch. The player may request the putting strategy information (voice guidance information) by pressing the button switch. In response to the player's press of the button switch, the input unit 410 may provide the controller with a control signal requesting the putting strategy information (voice guidance information).

In various embodiments, the input unit 410 may also be implemented as a pressure sensor which senses pressure on a grip surface. For example, the input unit 410 may sense the pressure exerted by the player gripping the grip, input through the grip surface. In an embodiment, the input unit 410 may be implemented as the pressure sensor described with reference to FIGS. 2 and 3. The input unit 410 may sense the pressure exerted by gripping the grip and transmit the sensed pressure to the controller 440.

The controller 440 may control the operation of the putting support device 400. The controller 440 may be the MCU described with reference to FIGS. 2 and 3.

The controller 440 may receive the control signal requesting the putting strategy information (voice guidance information) from the input unit 410. Based on the received control signal, the controller 440 may control the communication unit 420 to acquire location information of the ball and provide the acquired location information of the ball to the terminal 100.

In an embodiment, when the input unit 410 is implemented as a pressure sensor, the controller 440 may determine whether the player has taken the address position based on the pressure signal received from the input unit 410. When the controller 440 determines that the player has taken the address position, the controller 440 may control the communication unit 420 to calculate the location information of the ball, which is current location information. For example, the controller 440 may determine that the player has taken the address position when the pressure signal of the player falls within a preset range. The controller 440 may determine that the player has not taken the address position when the pressure signal of the player falls outside the preset range.

In an embodiment, the communication unit 420 may include the GPS antenna, the precision GPS module (RTK module), and the communication module described with reference to FIGS. 2 and 3. The controller 440 may control the GPS antenna or the precision GPS module (RTK module) of the communication unit 420 to acquire location information and control the communication unit 420 to provide the acquired location information, i.e., the location information of the ball, to the terminal 100.

The terminal 100 may provide the location information of the ball and player information received via the communication unit 420 to the server 200. The server 200 may generate the putting strategy information based on the received information and provide the putting strategy information to the terminal 100. The terminal 100 may extract the voice guidance information from the putting strategy information or generate the voice guidance information based on the putting strategy information. In an embodiment, the putting strategy information may include information regarding putting strength and putting direction. The putting strength may be the strength reflecting the distance from the location of the ball to the hole cup and the slope of the green. For example, when the hole cup is 10 meters away but on an uphill slope, a stronger strength than 10 meters may be indicated. The putting direction may be directional information indicating a point the player should aim for relative to the hole cup, considering the influence of the slope to the left and right of the hole cup on a path of the ball. The voice guidance information may be information for providing the player with information regarding putting strength and putting direction via voice. The terminal 100 may provide the voice guidance information to the putting support device 400.

The output unit 430 may output the voice guidance information received via the communication unit 420 to the player. To output the voice guidance information, the output unit 430 may include the speaker described with reference to FIGS. 2 and 3.

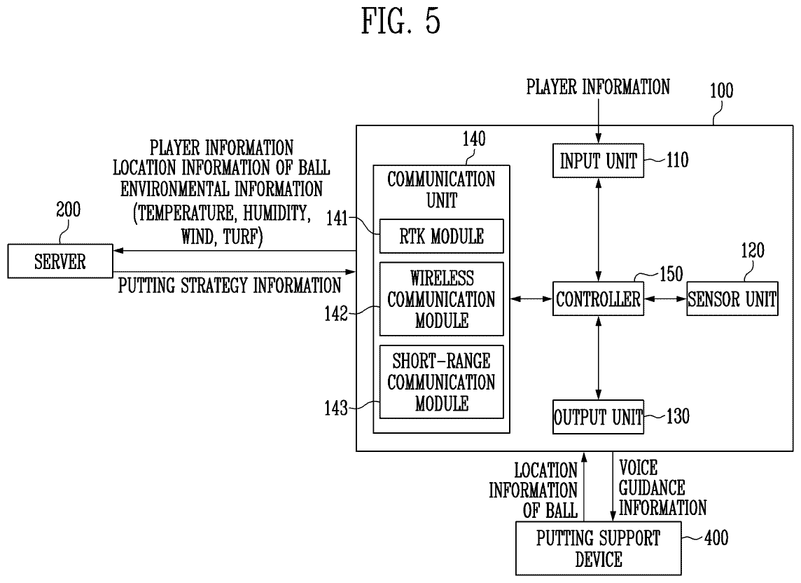

FIG. 5 is a diagram illustrating an operation of the terminal 100.

Referring to FIG. 5, the terminal 100 may include an input unit 110, a sensor unit 120, an output unit 130, and a communication unit 140. The communication unit 140 may include a real-time kinematic (RTK) module 141, a wireless communication module 142, and a short-range communication module 143.

The input unit 110 may receive input from the player. The input unit 110 may include input interfaces such as a touch panel, touch screen, touch pad, touch ball, camera, microphone, gyroscope sensor, vibration sensor, or piezoelectric element.

The input unit 110 may receive player information from the player. The player information may be information such as an ID and password for identifying the player.

The sensor unit 120 may include a temperature sensor or humidity sensor for sensing environmental information (e.g., wind, temperature, humidity, and the like) of the golf course.

The output unit 130 may include output interfaces such as a Liquid Crystal Display (LCD), an Organic Light Emitting Diode (OLED) display device, an Active Matrix OLED (AMOLED) display device, an LED display device, a speaker, or a monitor.

The communication unit 140 may include the RTK module 141, the wireless communication module 142, and the short-range communication module 143.

The RTK module 141 may be a GPS module to which the concept of RTK positioning is applied, and may acquire precise positioning results in real time at a rover by using correction values for the carrier phase of a base station (GPS base) possessing precise location information. While conventional GPS modules may incur various errors due to factors like the ionosphere, atmosphere, and satellite errors, the RTK module 141 may compensate for the degradation in positioning accuracy caused by signal distortion and delays occurring when GPS satellite signals pass through the ionosphere, thereby reducing positioning errors to as little as 1 to 2 cm. In an embodiment, the RTK module 141 may receive RTK correction signals from nearby base stations using a Virtual Reference System (VRS) method, a Flachen Korrektur Parameter (FKP) method, a Master Auxiliary Concept (MAC) method, or a State Space Representation (SSR) method.

Because the putting support device 400 in FIG. 4 includes the RTK module, the putting support device 400 may provide the terminal 100 with location information corrected by the RTK module. Alternatively, in various embodiments, the putting support device 400 may include only a GPS module. When the putting support device 400 provides the terminal 100 with location information acquired by the GPS module, the RTK module 141 in the terminal 100 may correct the precise location of the putting support device 400 based on the distance between the terminal 100 and the putting support device 400.

In various embodiments, at least two RTK modules may be located around the green of each hole on the golf course. The terminal 100 might not acquire the location of the ball (that is, the location of the putting support device 400), and the putting support device 400 may instead acquire the location information of the ball using at least two RTK modules located around the green. For example, the putting support device 400 described with reference to FIG. 4 may receive GPS signals and correct the GPS signals using at least two RTK modules located around the green. The putting support device 400 may receive RTK correction signals from at least two RTK modules located around the green using a Virtual Reference System (VRS) method, a Flachen Korrektur Parameter (FKP) method, a Master Auxiliary Concept (MAC) method, or a State Space Representation (SSR) method. In an embodiment, the putting support device 400 may also acquire the location information of the ball using a triangulation method based on the location information of at least two RTK modules located around the green.

The wireless communication module 142 may connect the terminal 100 to a wireless communication network for communication with the server 200 and may be implemented in various methods, such as LTE or 5G, depending on a protocol.

The short-range communication module 143 is a module for communicating with the putting support device 400 and may provide various short-range wireless communication interfaces such as Bluetooth, Wi-Fi, ZigBee, or Ultra-Wideband (UWB) communication method. The short-range communication module 143 may provide the voice guidance information to the putting support device 400 based on the putting strategy information received from the server 200.

A controller 150 may be a processor which controls the overall operation of the terminal 100. The controller 150 may control the input unit 110, the sensor unit 120, the output unit 130, and the communication unit 140. In an embodiment, the controller 150 may provide the player information received via the input unit 110, environmental information (e.g., wind, temperature, humidity, and the like) acquired via the sensor unit 120, and the location information of the ball acquired via the short-range communication module 143 to the server 200 via the wireless communication module 142 of the communication unit 140. In an embodiment, the controller 150 may receive the putting strategy information from the server 200 via the wireless communication module 142 and output the putting strategy information via the output unit 130 to provide the putting strategy information to the player.

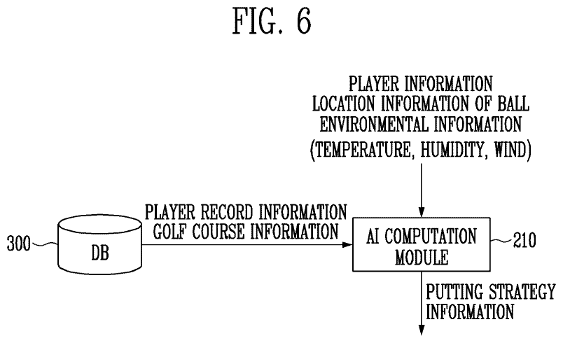

FIG. 6 is a diagram illustrating an operation of the server 200.

Referring to FIG. 6, the AI computation module 210 of the server 200 may receive the player information, location information of the ball, and the environmental information from the terminal 100. The AI computation module 210 may acquire previously stored player record information from the DB 300. The player record information may include the average number of putts, putting distance information, putting trajectory information, and the like, learned from the past play results of the player. The AI computation module 210 may acquire golf course information from the DB 300. The golf course information may include terrain information of the golf course, par for each hole, slope of the green, green speed information, pin location for each hole, and turf information of the golf course.

The green speed information may be data provided by the golf course. The server 200 may receive the green speed information from the golf course via a remote network. The green speed information may represent the speed of the green as a numerical value. The green speed may be acquired by measuring the speed back and forth 3 to 5 times on a flat green surface and dividing the average value by feet. For example, the green speed may be the numerical value representing the average distance traveled by a ball when placed on a Stimpmeter of which a horizontal surface is located on the green and which is tilted at a 20-degree angle. A higher green speed number indicates a faster green.

The AI computation module 210 may generate the putting strategy information based on the golf course information (particularly, using the slope of the green and green speed) and the location information of the ball. The putting strategy information may include information regarding putting strength and putting direction. The putting strength may reflect the distance from the location of the ball to the hole cup and the slope of the green. The putting direction may represent directional information indicating the point the player should aim for relative to the hole cup, considering the influence of slope to the left and right of the hole cup on the path of the ball.

In an embodiment, the AI computation module 210 may generate the putting strategy information optimized for the player based on the player record information. For example, the AI computation module 210 may generate the putting strategy information reflecting the average number of putts, putting distance information, and putting trajectory information included in past records of the player.

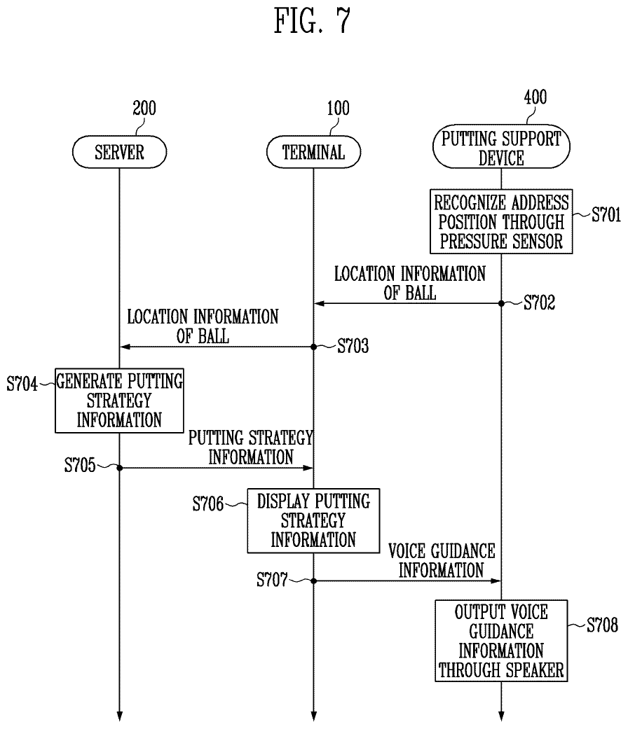

FIG. 7 is a diagram illustrating an operating method of a putting support system according to an embodiment.

Referring to FIG. 7, in step S701, the putting support device 400 may recognize the address position of the player through the pressure sensor.

In step S702, the putting support device 400 provides the location information of the ball, acquired using the GPS module or RTK module, to the terminal 100. In step S703, the terminal 100 may provide the location information of the ball to the server 200.

In step S704, the server 200 generates the putting strategy information based on the golf course information and the location information of the ball. In step S705, the server 200 may provide the generated putting strategy information to the terminal 100.

In step S706, the terminal 100 may display the received putting strategy information.

In step S707, the terminal 100 may provide the voice guidance information to the putting support device 400.

In step S708, the putting support device 400 may output the voice guidance information to the player through a speaker.

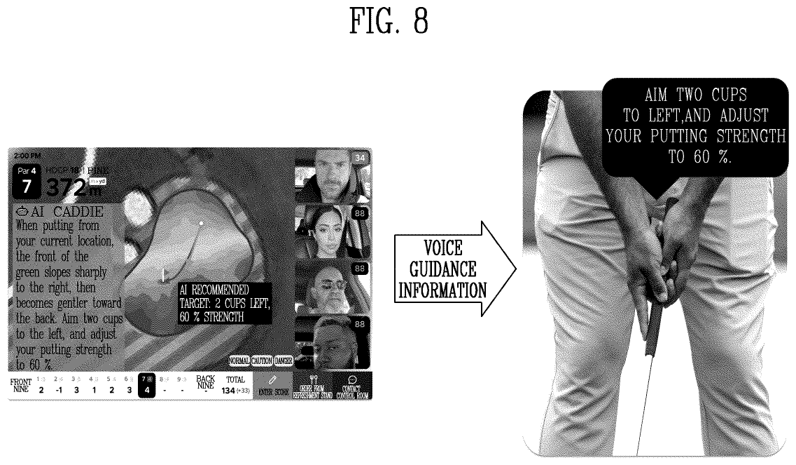

FIG. 8 a diagram illustrating an example of a putting strategy provided to the player by the terminal 100 and the putting support device 400.

Referring to FIG. 8, the output unit 130 of the terminal 100 may provide the location of the ball, the location of the hole cup, the slope of the green, an expected direction of travel of the ball, and the putting strategy information.

Specifically, referring to the putting strategy information from an AI caddie, the advice provided is to aim two cups to the left, which is more left of the expected direction of travel because the slope sharply inclines to the right before becoming gentle when facing the hole cup direction from the current location of the ball, and putt with 60 % of strength because it is a downhill putt.

The speaker of the putting support device 400 may output the voice guidance information instructing the player to “aim two cups to the left and adjust the putting strength to 60 %.”

According to the putting support system according to an embodiment of the present disclosure as described above, the putting support system may acquire the most accurate location of the ball in real time by using the putting support device mounted on the grip of the putter and detecting when the player takes the address position, thereby providing the player with precise putting strategy information.

The present disclosure is directed to a system which provides real-time putting guidance to a golf player through the putting support device 400 mounted on the putter. The putting support device 400 may include the GPS antenna, the RTK module, the pressure sensor, and the speaker. Specifically, the MCU of the putting support device 400 may be implemented using a dedicated System on Chip (SoC). Additionally, adding an LTE/Wi-Fi module resolves the distance limitations inherent in low-power Bluetooth methods. Furthermore, through low-power design and miniaturization, battery capacity may be reduced, enabling further weight reduction.

Although the present disclosure has been described with reference to specific embodiments and drawings, the present disclosure is not limited to these embodiments. Those skilled in the art to which the present invention pertains may make various modifications and variations based on the above description.

Therefore, the scope of the present disclosure should not be limited to the described embodiments, but should be defined by the appended claims and equivalents thereof.

In the above-described embodiments, all steps may be performed selectively or omitted. Furthermore, in each embodiment, the steps need not necessarily occur in sequence and may be reversed. The embodiments disclosed in this specification and the drawings are merely specific examples provided to readily explain the technical content of this specification and aid in its understanding, and are not intended to limit the scope of this specification. That is, it is obvious to those skilled in the art to which this specification pertains that other variations based on the technical spirit of this specification are feasible.

The present specification and drawings disclose preferred embodiments of the present disclosure. Although specific terms are used, they are employed in a general sense to facilitate explanation of the technical content and aid understanding of the present disclosure, and are not intended to limit the scope of the present disclosure. It is obvious to those skilled in the art to which the present invention pertains that other variations based on the technical spirit of the present invention are possible beyond the embodiments disclosed herein.

Embodiments of the present disclosure may provide a putting support system and an operating method thereof.

What is claimed is:

1. A putting support system comprising:
a putting support device embedded in a grip of a putter of a player, detecting an address position of the player on a green, and acquiring and outputting location information of a ball, which is location information at a time when the address position is detected;
a server generating putting strategy information for a location of the ball based on the location information of the ball, slope of the green, and location information of a hole cup; and
a terminal receiving the location information of the ball from the putting support device, transmitting the location information of the ball to the server, receiving the putting strategy information from the server, and providing the putting support device with voice guidance information determined according to the putting strategy information.

2. The putting support system of claim 1, wherein the putting support device comprises:
a sensor unit sensing a pressure signal transmitted to the grip of the putter; and
a controller detecting the address position of the player based on the pressure signal.

3. The putting support system of claim 2, wherein the controller determines that the player has taken the address position when the pressure signal falls within a preset range.

4. The putting support system of claim 3, wherein the putting support device further comprises a communication unit acquiring location information of the putting support device using real-time kinematic (RTK) positioning in response to the controller determining that the player has taken the address position.

5. The putting support system of claim 4, wherein the communication unit communicates with the terminal via a short-range communication method.

6. The putting support system of claim 1, wherein the putting support device further comprises a speaker providing the voice guidance information to the player.

7. The putting support system of claim 1, wherein the putting support device further comprises:
a rechargeable battery providing driving power to the putting support device; and
a charging terminal charging the rechargeable battery via a wired or wireless method.

8. The putting support system of claim 1, wherein the putting strategy information includes information regarding putting strength and putting direction.

9. The putting support system of claim 1, wherein the terminal is mounted on a golf cart in which the player rides.

10. The putting support system of claim 1, further comprising a database storing player record information including previous play information of the player and golf course information including the slope of the green and the location information of the hole cup.
